(19) United States
(12) Patent Application Publication
(10) Pub. No.: US 2024/0230571 A1
(43) Pub. Date: Jul. 11, 2024

(54) VOLATILE COMPOUND SOURCE LOCATING SYSTEMS

(71) Applicant: University of Utah Research Foundation, Salt Lake City, UT (US)

(72) Inventors: Hanseup KIM, Salt Lake City, UT (US); Seungbeom NOH, Salt Lake City, UT (US); Shakir KHAN, Salt Lake City, UT (US); Ling ZANG, Salt Lake City, UT (US); Carlos H. MASTRANGELO, Salt Lake City, UT (US)

(21) Appl. No.: 18/407,655

(22) Filed: Jan. 9, 2024

Related U.S. Application Data
(60) Provisional application No. 63/437,898, filed on Jan. 9, 2023.

(51) Int. Cl. G01N 27/12; A01G 7/00; G01N 33/00

(52) U.S. Cl. CPC G01N 27/125; A01G 7/00; G01N 27/122; G01N 33/0047

(57) ABSTRACT

A volatile compound source locating system can include an array of chemical sensors distributed in a detection volume. The individual chemical sensors can include a positive electrode, a negative electrode separated from the positive electrode by a switch gap, and a binding agent located at a plurality of binding sites in the switch gap. The binding agent can be selective for binding to a target volatile compound. The binding sites can be capable of binding molecules of the target compound to form an electrically conductive pathway between the positive and negative electrode when the chemical sensor is exposed to a threshold concentration of the target compound. A source locating module can be in electronic communication with the array of chemical sensors, and configured to estimate the location of the source of the target compound based on electronic signals from the array of chemical sensors.

### CROSS REFERENCE TO RELATED APPLICATIONS

This application claims priority to U.S. Provisional Application No. 63/437,898, filed Jan. 9, 2023 which is incorporated herein by reference.

### STATEMENT REGARDING FEDERALLY SPONSORED RESEARCH OR DEVELOPMENT

This invention was made with government support under grant DE-AR0001064 awarded by the Department of Energy. The government has certain rights in this invention.

### NAMES OF THE PARTIES TO A JOINT RESEARCH AGREEMENT

Not applicable.

### INCORPORATION BY REFERENCE STATEMENT

Not applicable.

### BACKGROUND

Existing chemical sensors include conductivity, piezoelectric, optical, and field-effect-transistor (FET) sensors. To continuously monitor for target chemicals, these sensors often consume significant amounts of electric power. Thus, these sensors are not ideal for operating on battery power for a long period of time. Some types of chemical sensors also operate at high temperatures. These cannot be used to monitor for chemicals in the ambient atmosphere at normal ambient temperatures. Thus, many types of sensors are not suitable for continuous monitoring of ambient air to detect airborne chemicals.

### SUMMARY

This invention relates to volatile compound source locating systems and methods of locating sources of volatile compounds over a long period of time. In one example, a volatile compound source locating system includes an array of chemical sensors distributed in a detection volume. The individual chemical sensors can include a positive electrode, a negative electrode separated from the positive electrode by a switch gap, and a binding agent located at a plurality of binding sites in the switch gap. The binding agent can be selective for binding to a target volatile compound. The binding sites can be distributed in the switch gap such that the binding sites are capable of binding molecules of the target volatile compound to form an electrically conductive pathway via percolation between the positive electrode and the negative electrode when the chemical sensor is exposed to a threshold concentration of the target volatile compound. The switch gap provides additional selectivity beyond chemistry matching as it allows the electrical bridging when the captured molecules precisely match the size of the gap for complete physical connection. The system also includes a source locating module in electronic communication with the array of chemical sensors. The source locating module can be configured to estimate a location of a source of the target volatile compound based on electronic signals from the array of chemical sensors.

In various examples, the target volatile compound may be a volatile organic compound released by a plant, or a disease biomarker released by an animal, or a chemical weapon. In some specific examples, the target volatile compound can be hexanal, hexenal, hexenol, acetaldehyde, decanal, or a combination thereof.

The individual chemical sensors can include a plurality of electrically conductive structures in the switch gap. The electrically conductive structures can be planar islands, vertical pillars, or horizontal parallel plates formed in the switch gap. In some examples, the planar islands, vertical pillars, or horizontal plates can have a shape selected from circular, hexagonal, square, rectangular, and triangular. In certain examples, the vertical pillars can have a width from 5 nanometers to 100 micrometers and the horizontal parallel plates can have a width from 1 micrometer to 1 millimeter. In one example, the electrically conductive structures can be planar islands having the binding agent attached to surrounding edges of the planar islands. In another example, the electrically conductive structures can be vertical pillars having the binding agent attached to vertical surfaces of the vertical pillars. In yet another example, the electrically conductive structures can be horizontal parallel plates having the binding agent attached to horizontal surfaces of the horizontal parallel plates.

In a specific example, the target volatile compound can be hexanal and the horizontal plates can be separated one from another by structure gaps having a structure gap distance from 5 nanometers to 6 nanometers. In other examples, the electrically conductive structures can be separated one from another by structure gaps having a structure gap distance from 0.3 nanometer to 100 micrometers.

Non-limiting examples of the binding agent can include conductive molecules custom-designed or commercially available, such as, but not limited to, thiol-functional-group to bind hexanal-functional-group chemicals, aldehyde- or hydroxyl-functional-group to bind carboxyl-functional-group chemicals, hydrophobic interaction to bind the alkane-functional-group chemicals, and hydrogen-bonding to bind ester-functional-group chemicals, in some examples. Note that these binding groups can be formed at the end of a backbone structure that can have multiple forms to maintain conductivity and rigidness of a linker.

The individual chemical sensors can be spaced apart one from another at an array spacing from about 1 meter to about 100 meters. The individual chemical sensors can also include an amplifier in some examples. In further examples, the individual chemical sensors can include a microcontroller. In still further examples, the individual chemical sensors can include a battery.

The detection volume can contain a field of crops in certain examples. The array of chemical sensors can be located such that at least one of the chemical sensors is exposed to the target volatile compound when released from the crops. The field of crops can include sorghum, wheat, corn, soybean, potatoes, rice, nuts, cotton, vegetables, and fruits, or a combination thereof.

The source locating module can be in electronic communication with the array of chemical sensors through wireless data transmission. In some examples, the system can also include a wind sensor in electronic communication with the source locating module.

The present disclosure also describes methods of locating a source of a volatile compound. In one example, a computer implemented method of locating a source of a volatile compound includes: receiving an electronic signal from at least one chemical sensor of an array of chemical sensors distributed in a detection volume, wherein individual chemical sensors comprise a positive electrode, a negative electrode separated from the positive electrode by a switch gap, and a binding agent located at a plurality of binding sites in the switch gap, wherein the binding agent is selective for binding to a target volatile compound, wherein the binding sites are distributed in the switch gap such that the binding sites are capable of binding molecules of the target volatile compound to form an electrically conductive pathway via percolation between the positive electrode and the negative electrode when the chemical sensor is exposed to a threshold concentration of the target volatile compound; and estimating a location of a source of the target volatile compound using the electronic signal from the at least one chemical sensor and a propagation model of the target volatile compound, wherein the electronic signal is a parameter input into the propagation model. In certain examples, the electronic signal can be received from multiple chemical sensors that detected the threshold concentration of the target volatile compound, and the locations of the multiple chemical sensors can be parameters input into the propagation model. In a further example, the multiple chemical sensors can detect the threshold concentration at multiple detection times, and the multiple detection times can also be a parameter input into the propagation model. In another example, wind speed and wind direction can be parameters input into the propagation model. The method can also include measuring the wind speed and the wind direction within the detection volume.

The individual chemical sensors can draw standby power when not exposed to the threshold concentration of the target volatile compound. The standby power can be from 1 mW to 100 mW.

The present disclosure also describes methods of mitigating pest damage to crops. In one example, a method of mitigating pest damage to crops can include: detecting a volatile organic compound released by a damaged crop by receiving an electronic signal from at least one chemical sensor of an array of chemical sensors distributed in a crop field, wherein individual chemical sensors comprise a positive electrode, a negative electrode separated from the positive electrode by a switch gap, and a binding agent located at a plurality of binding sites in the switch gap, wherein the binding agent is selective for binding to the volatile organic compound, wherein the binding sites are distributed in the switch gap such that the binding sites are capable of binding molecules of the volatile organic compound to form an electrically conductive pathway via percolation between the positive electrode and the negative electrode when the chemical sensor is exposed to a threshold concentration of the volatile organic compound; estimating a location of the damaged crop using the electronic signal from the at least one chemical sensor and a propagation model of the volatile organic compound, wherein the electronic signal is a parameter input into the propagation model; and applying a pesticide at the estimated location. In various examples, the crops can include sorghum, wheat, com, soybean, potatoes, rice, nuts, cotton, vegetables, and fruits, or a combination thereof.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a schematic illustration of a volatile compound source locating system in accordance with one example.

FIG. 2A-2C are three schematic illustrations of example arrangements of chemical sensors arrays in accordance with several examples.

FIG. 3 is a schematic illustration another volatile compound source locating system in accordance with another example.

FIG. 4 is a side cross-sectional view of a single-gap chemical sensor in accordance with another example.

FIGS. 5A-5C are top-down schematic views of an array of chemical sensors for percolation-based switching in accordance with another example.

FIG. 6A is a cross-sectional side view of another example chemical sensor in accordance with another example.

FIG. 6B is a top-down view of the chemical sensor shown in FIG. 5A.

FIG. 7 is a schematic illustration of a chemical sensor prototype including interface circuits in accordance with an example of the present disclosure.

FIG. 8 is a flowchart illustrating an example computer implemented method of locating a source of a volatile compound in accordance with an example of the present disclosure.

FIG. 9 is a flowchart illustrating a method of mitigating pest damage to crops, in accordance with another example.

FIG. 10 is a schematic illustration of a test system for detecting hexanal in accordance with another example.

FIG. 11 is an algorithm flowchart for wireless communication of a chemical sensor in accordance with another example.

FIG. 12 is an algorithm flowchart for an example algorithm to be performed by a volatile compound source locating system in accordance with another example.

FIG. 13 is another algorithm flowchart for an example algorithm to be performed by a volatile compound source locating system in accordance with another example.

FIG. 14A is a schematic of a nanogap sensor having a top electrode, bottom electrode and corresponding bond pads with a vertical nanogap between the top electrode and bottom electrode in accordance with another example.

FIG. 14B is a schematic illustration of the vertical nanogap of FIG. 14A showing uncaptured and captured states of the sensor nanogap in accordance with an example.

FIG. 14C is a design schematic of the chemical sensor in accordance with an example.

FIG. 14D is an SEM image of a chemical sensor nanogap formed using the schematic of FIG. 14A-C.

FIG. 14E is an image of the chemical sensor formed using the schematic of FIG. 14A-C in accordance with another example.

FIG. 15 is a graph of sensor output signal change with change in temperature, in accordance with another example.

FIG. 16A is a schematic of test equipment for testing a chemical sensor in accordance with another example.

FIG. 16B is a graph of resistance over time of a chemical sensor in accordance with another example.

FIG. 16C shows gas chromatography mass spectrometer spectra of hexanal in accordance with another example.

FIG. 17 is a graph of sensor output signal change with change in humidity, in accordance with another example.

FIG. 18 is a graph of sensor off/on ratio with change in the concentration of hexanal, in accordance with another example.

FIG. 19 is a graph of distance at which hexanal was detected vs. number of cuts sorghum, in accordance with another example.

FIG. 20A shows a prototype source locating system associated with a portion of a crop in accordance with another example.

FIG. 20B is a graph of sensor response as a function of time for the example system of FIG. 20A.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a compound” includes reference to one or more of such materials and reference to “the sensor” refers to one or more of such sensors.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Example Embodiments

A technology is described for locating a source location of volatile compounds that are released into a particular area. This technology can allow continuous monitoring of an area for the release of a target volatile compound into the air. An array of chemical sensors can be distributed across the area of interest, which can be referred to as the “detection volume.” The chemical sensors, normally-off, can be activated by the presence of the target volatile compound in an amount that is above a threshold concentration. When one or more of the chemical sensors is activated, the chemical sensors can send electronic signals to a source locating module and the source locating module can estimate the location of the source of the target volatile compound based on the electronic signals from the chemical sensors.

Despite the recent advancement of sensors for crop monitoring, there is a lack of a holistic model that is capable of combining both environmental and real-time sensor measurement data and ultimately producing accurate prediction of the crop damage location, predicted spread over time and estimated economic loss at each time point. This is mainly due to the lack of either field-deployable or off-grid sensor network, a multi-physics prediction model of the spread of damages, and an associated economic model to compute the loss of yield, production amounts and economic values.

Incorporation of a localization model with a sensor network can help determine and optimize the yield loss, production efficiency and cost and labor for biofuel crop farm management. Farm management is typically carried out based on statistical data and personal experience, and the optimized management has been lacking. The recovery of biofuel crop loss has not been fully achieved despite sporadic deployment of various sensors in the field. This is because the crop damage and its spread depend on environments, types of causes, timeliness, and types and areas of treatments. For example, it is estimated that the treatment area can be reduced below 10% even down to 1% or below, depending on the size of the whole farm, when a computational model can narrow the damage onset points and spread speed.

The systems and methods described herein can ‘fill the gap’ of the technical vacuum toward ‘smart farming’ by the development of a holistic localization model that combines physical phenomena, environmental factors and economic analysis into a precise prediction of damage and economic loss over wireless tethering to individual smartphones. This model can serve as a bridge from the state-of-the-art field scouting system, to the demands for autonomous, high-efficiency, and thus economical biofuel crop production. The systems and methods can also involve other core field conditions such as humidity/irrigation, pH, and temperature, and enable a finely-tuned smart-farming solution.

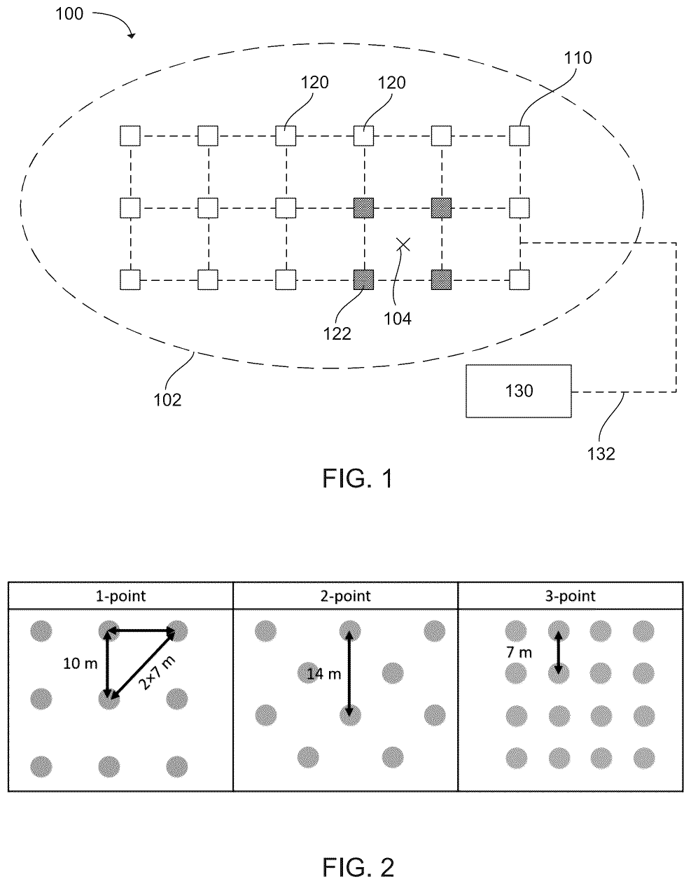

FIG. 1 illustrates a schematic top-down view of an example volatile compound source locating system 100. This system includes an array 110 of chemical sensors 120 distributed in a detection volume 102. A source locating module 130 is in electronic communication with the array of chemical sensors. In this figure, the electronic communication is represented by dashed lines 132 connecting the source locating module and the chemical sensors. The electrical connection can be wired or wireless in various examples. The source locating module is configured to estimate the location 104 of a source of the target volatile compound based on electronic signals from the array of chemical sensors. In the example shown, the source locating module receives a signal from four activated chemical sensors 122, shown as shaded boxes in the figure. The source locating module then estimates that the location of the source of the target volatile compound is between these four chemical sensors. Connection between the source locating module and the array can be direct (i.e. real time) or can be intermittent such that a temporary memory storage device can collect information from the array which is stored for a given time and then transferred to the source locating module 130. This may be useful for remote locations where data can be collected for a period of time (i.e. days, weeks or months) and then periodically collected to process the data to determine presence and source of the volatile compound.

FIGS. 2A-2C shows schematic diagrams of three additional example arrangements for a chemical sensor array. The first arrangement of FIG. 2A has chemical sensors arranged in aligned rows with 10 meter spaces between adjacent chemical sensors. The second arrangements of FIG. 2B has chemical sensors in staggered rows with 14 meter spaces between adjacent chemical sensors in the same row. The third arrangement of FIG. 2C has chemical sensors in aligned rows with 7-meter spaces between adjacent chemical sensors. Of course, the distance between sensors can be varied considerably depending on the desired sensitivity, resolution, and other factors.

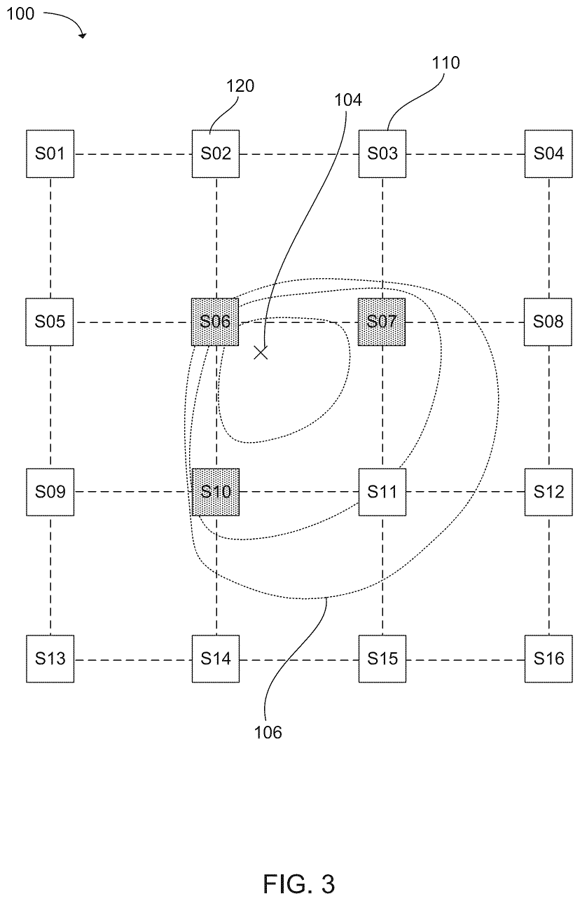

FIG. 3 is a schematic top-down view of another example volatile compound source locating system 100. This example includes sensors 120 labeled S01 through S16 arranged in a 4×4 array 110. A source location 104 is in the central area of the array. Three dotted lines 106 represent different volumes with different concentrations of the target volatile compound at three different times (t1, t2, and t3). In this example, sensors S06, S07, and S10 turn on because they detected a concentration of the target volatile compound above a threshold concentration. The sensors can turn on at different times, depending on when the target volatile compound reaches the sensors. The turn-on times of the individual sensors can be taken into account by the source locating module when estimating the source location. For example, volatile compound emanating from source 104 would disperse toward surrounding sensors at a rate which is a function of concentration, temperature, humidity, wind speed, and wind direction, among other factors. In the example, illustrated in FIG. 3, at time t1, sensor S06 would be triggered (assuming a threshold concentration is reached). At time t2, sensors S07 and S10 would also be triggered along with sensor S06.

In some cases, when a sensor is turned on, it can produce variable output signals depending on the concentration of volatiles as a higher concentration of volatiles produces an increased number of bridges instantly leading to a higher current output. The variable level of output signals can be utilized to estimate the distance from a source to the detecting sensor because it is correlated with the dilution in volatile concentration over a distance due to diffusion over a volume. This dilution in concentration can vary depending on the environmental conditions like wind speed. Hence localization testing can be conducted to locate the damage start point.

In certain examples, the source locating systems can be used to localize plant damage caused by herbivores in an early stage. The system can include a low-power gas sensor array in a field of plants. Using such a system can result in recovering crop loss due to herbivore attacks and the limited capabilities of current scouting methods, reducing farm management labor and cost, and leading to autonomous smart farming. The array of low-power gas sensors can monitor for signature gas molecules. In one example, the sensors can monitor for hexanal released by mechanically damaged sorghum plants. The sensors can wirelessly transfer detection data to a source locating module at a central station. The combined data from the sensors can then be used to calculate the location of the plant damage. The calculation can be performed by combining the detection data with environmental contributors such as temperature, humidity, wind direction, wind speed, etc. Thus, the source of the plant damage can be located and the system can alert users such as farmers to provide a field treatment. This can reduce crop loss and prevent late field treatment, over-spraying of pesticides at healthy crops, and can eventually lead to autonomous smart farming without intensive labor and cost.

The chemical sensors used in the present technology can be selective for the target volatile compound using both physical size screening and chemical screening using binding agents that selectively bind to the target volatile compound. In some examples, the chemical sensors can include a nanogap between two electrically conductive structures and molecules of a binding agent attached to the electrically conductive structures. The nanogap can be sized so that a single molecule of the target volatile compound can be bound between binding agent molecules on one or both sides of the nanogap. The bound molecule of the target volatile compound can provide a pathway for electric current to pass across the nanogap, thus causing a measurable change in electrical resistance across the nanogap.

The chemical sensors can also be designed to utilize percolation to form an electrically conductive pathway when the chemical sensors are exposed to a threshold concentration of the target volatile compound. In some examples, the chemical sensors can include an array of electrically conductive structures that are separated by nanogaps, with a binding agent on surfaces of the electrically conductive structures as described above. The electrically conductive structures can be located in a switch gap between a positive electrode and a negative electrode. When the nanogaps between the electrically conductive structures are empty, the resistance across the switch gap can be very high. Thus, even though an electric voltage may be applied to the positive and negative electrodes, no electric current flows and therefore the chemical sensor consumes no power or very little power. However, if a sufficient number of molecules of the target volatile compound occupy binding sites between the electrically conductive structures to form a continuous electrically conductive pathway, then electric current can flow between the positive electrode and negative electrode, which indicates that the chemical sensor has been exposed to the threshold concentration of the target volatile compound.

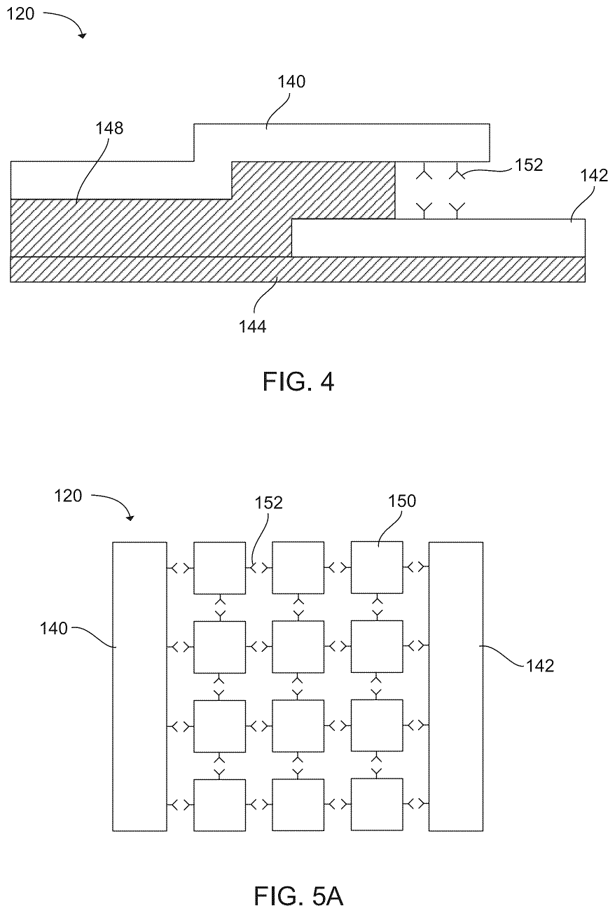

FIG. 4 shows a side cross-sectional view of one type of chemical sensor 120 that can be used in the systems described herein. This sensor includes an upper positive electrode 140 and a lower negative electrode 142. It is noted that these can be reversed in other examples, with the positive electrode being the lower electrode and the negative electrode being the upper electrode. This sensor does not utilize percolation and does not include any other electrically conductive structures between the positive electrode and the negative electrode. The positive electrode and negative electrode are separated by a nanogap that is sized to admit single molecules of a target volatile compound. The surfaces of the electrodes are treated with binding agent molecules 152 linked to the surfaces. The gap between the electrodes can ensure electrical separation between the electrodes with a negligible tunneling current between the electrodes even when a biasing voltage is applied. When a molecule of the target volatile compound is captured by the binding agent molecules, it can increase electrical conductivity across the nanogap. In this example, the negative electrode is formed by depositing a metal such as gold on a substrate 144. Of course, any suitable electrically conductive material can be used (e.g. copper, gold, silver, graphite, titanium, etc). A dielectric layer 148 can then be deposited over the negative electrode and the positive electrode can be deposited over the dielectric layer. The empty gap between the positive electrode and the negative electrode can be formed by depositing a sacrificial material over a portion of the negative electrode and then removing the sacrificial material after the positive electrode has been formed. Alternatively, a portion of the dielectric layer can be removed between the positive electrode and the negative electrode.

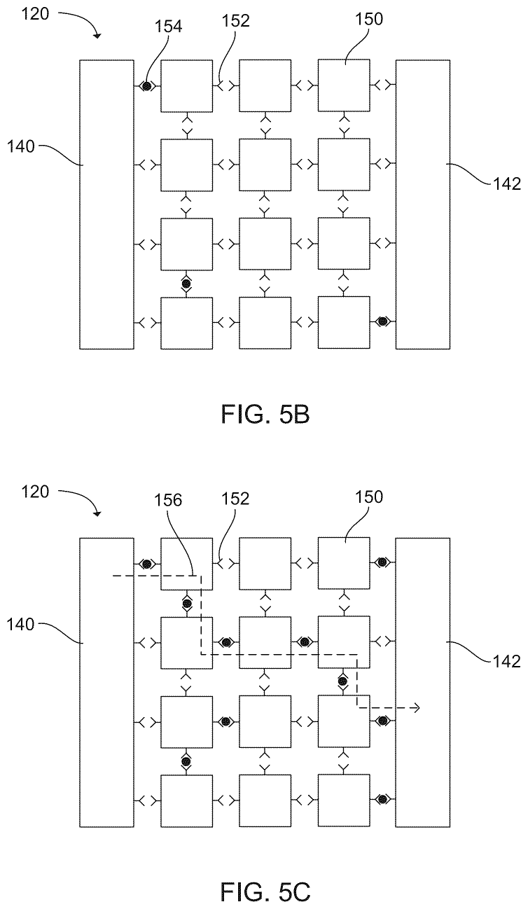

To clearly illustrate the operation of the chemical sensors described herein, an example chemical sensor 120 is shown in FIG. 5A which is a percolation type design. This sensor includes a positive electrode 140 and a negative electrode 142 separated by a switch gap. A plurality of electrically conductive structures 150 are oriented in the switch gap between the positive and negative electrodes. In this example, the electrically conductive structures are square-shaped pillars. The electrically conductive structures are separated one from another by nanogaps. It is noted that the term “nanogap” is used herein to refer to the gaps between electrically conductive structures that are sized to specifically allow a single molecule of the target volatile compound into the nanogap. Thus, the nanogaps typically have a gap size on the nanometer scale. The term “switch gap” is used to refer to the gap between the positive electrode and the negative electrode. The switch gap can have a much larger gap distance because the switch gap is large enough for multiple electrically conductive structures to be placed between the positive electrode and negative electrode, with nanogaps between each of the relevant electrically conductive structures Binding agent molecules 152 are attached to the surfaces of these electrically conductive structures. The binding agent molecules can be chemically selective for binding to the target volatile compound. Molecules of the target volatile compound can be bound to the binding agent molecules at certain locations to form electrically conductive bridges. These locations are referred to as “binding sites” herein. In FIG. 5A, the binding sites are illustrated as a point between two binding agent molecules where a target volatile compound molecule can be bound. It is noted that the sensor in this figure is shown with a single binding site in each nanogap between the electrically conductive square-shaped pillars. However, in practice there can be many molecules of the binding agent on the surfaces of the electrically conductive structures, and any place where a molecule of the target volatile compound can be bound to form an electrically conductive bridge can be referred to as a binding site.

In the example chemical sensor 120 shown in FIG. 5A, none of the binding sites are occupied by molecules of the target volatile compound. Therefore, there is no electrical conductivity or very low conductivity between the positive electrode 140, the electrically conductive structures 150, and the negative electrode 142. FIG. 5B shows the chemical sensor with a few molecules of the target volatile compound 154 occupying binding sites These molecules can form electrically conductive bridges between adjacent electrically conductive structures, i.e., the square-shaped pillars in this example, although other shaped may be used such as, but not limited to, triangle, square, hexagonal, parallelogram, etc. However, there are not enough molecules of the target volatile compound present to make a continuous conductive pathway from the positive electrode to the negative electrode. FIG. 5C shows the chemical sensor when exposed to a higher concentration of the target volatile compound. At a certain threshold concentration, the sensor is likely to have at least one continuous conductive pathway from the positive electrode to the negative electrode. In this example, the electrically conductive pathway is represented by a dashed line 156 that passes through several electrically conductive square-shaped pillars that have been connected by molecules of the target volatile compound.

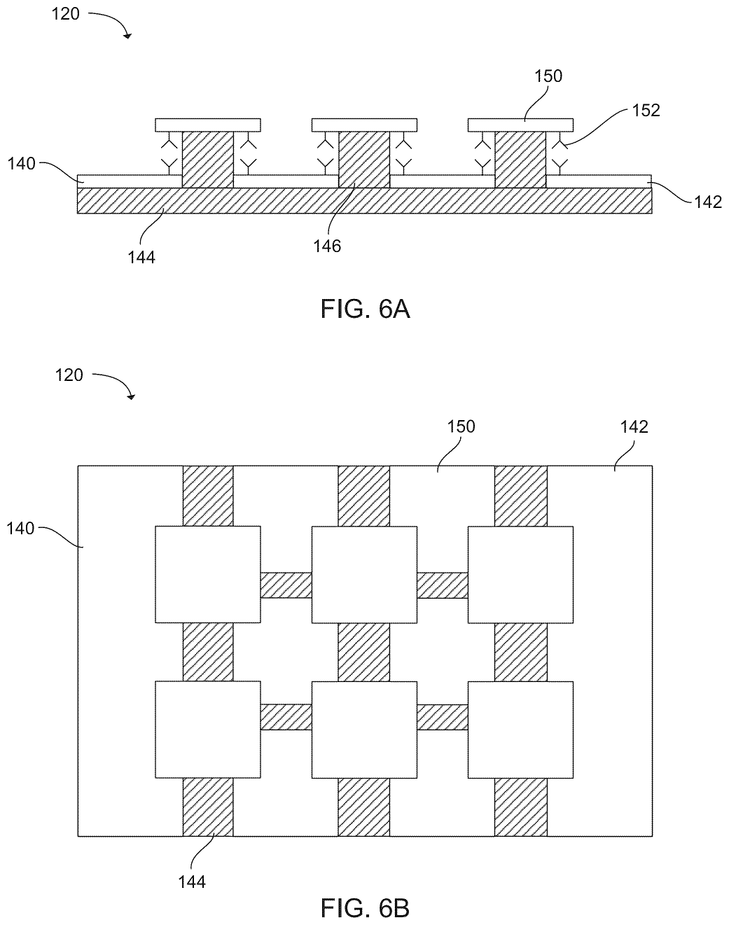

FIG. 6A is a cross-sectional side view of another example chemical sensor 120. This example also includes a positive electrode 140, a negative electrode 142, and a switch gap between the positive and negative electrodes. An array of electrically conductive structures 150 are positioned in the switch gap. In this example, the electrically conductive structures are square-shaped horizontal plates. The plates are arranged in an overlapping manner. A set of lower plates is deposited on a substrate layer 144. The positive and negative electrodes are also deposited on the substrate layer. A set of upper plates is formed on raised columns 146. The substrate and the raised columns can be made of dielectric materials. Portions of the upper plates overlap with portions of the lower plates. The upper plates are separated from the lower plates by a nanogap, and binding agent molecules 152 are attached to the surfaces of the upper and lower plates. The spaces between the binding agent molecules in the nanogaps act as binding sites. Thus, an electrically conductive pathway can be formed across the switch gap when molecules of the target volatile compound occupy the binding sites. FIG. 6B shows a top-down view of this example sensor. In this example, the upper plates and lower plates are arranged in a staggered manner so that each upper plate overlaps with multiple lower plates, and vice versa. Binding sites can be present in the nanogaps between all of the overlapping upper and lower plates. Thus, a variety of different electrically conductive pathways can form by percolation when the target volatile compound is present. More details regarding such percolation switch structures can be found in U.S. Pat. No. 10,502,725 which is incorporated herein by reference. Although percolation-based sensors can be particularly useful, any suitable chemical sensor can be used. Non-limiting examples of such sensors can include nanogap structure (e.g. vertical nanogap similar to FIG. 4, lateral nanogaps, etc).

The examples shown above include electrically conductive structures in the form of square-shaped pillars or square-shaped plates. A variety of other forms of electrically conductive structures can also be used, including planar islands, vertical pillars, horizontal parallel plates, and others. The structures can have a variety of shapes, such as circular, square, rectangular, triangular, hexagonal, or other shapes. The size of the electrically conductive structures can be adjusted depending on the desired size of the sensor. The surface area of the structures can also affect the amount of target volatile compound that can bind to binding agents on the surfaces, and this can affect the threshold concentration at which the sensor will detect the target volatile compound. Widths and dimensions can be chosen based on the particular target volatile compound and are not specifically limited. However, as a general guideline and in certain examples, the structures can be vertical pillars having a width from 5 nanometers to 100 micrometers, or from 10 nanometers to 10 micrometers, or from 50 nanometers to 1 micrometer, or from 50 nanometers to 500 nanometers. In other examples, the structures can be horizontal plates having a width from 1 micrometer to 1 millimeter, or from 1 micrometer to 500 micrometers, or from 10 micrometers to 500 micrometers, or from 10 micrometers to 50 micrometers.

The electrically conductive structures can be separated one from another by nanogaps, also referred to as “structure gaps.” The binding agent can be attached to the surfaces facing one another across the structure gaps. In some examples, the structures can be planar islands and the binding agent can be attached to surrounding edges of the planar islands. In other examples, the structures can be vertical pillars and the binding agent can be attached to vertical surfaces of the vertical pillars. In still other examples, the structures can be horizontal plates and the binding agent can be attached to horizontal surfaces of the horizontal plates.

In some examples, the binding agent can bind with the target volatile compound through a reversible bond, such as hydrogen bonding. Therefore, molecules of the target volatile compound may bind with the binding agent temporarily and then be released some time later. The threshold concentration that will cause a continuous electrically conductive pathway to form between the positive and negative electrodes can depend on several factors, including the kinetics of the target volatile compound binding to and being released from the binding agent. The longer the target volatile compound occupies binding sites in the sensor, the more likely it will be that a continuous electrically conductive pathway will form between the positive and negative electrodes. The threshold concentration can also depend on the number and arrangement of the electrically conductive structures in the switch gap and the number of binding agent molecules present in the nanogaps between the electrically conductive structures. Thus, the threshold concentration can be tuned by adjusting these parameters.

The systems described herein can be useful for detecting a variety of different volatile compounds. In particular, the systems can be useful when it is desired to find the source of the volatile compound, or in other words, the location where the volatile compound was released into the air. Some applications in which the systems can be used include finding the location of damaged crops by detecting a volatile organic compound released by damaged crops, or finding a diseased animal by detecting a volatile biomarker of the disease, or locating a chemical weapon source. In a more specific example, a system as described herein can be used to mitigate crop damage due to pests such as insects. The timing and location of crop damage can be difficult to identify. Manual scouting for crop damage involves being physically present at the location of the damaged crops in a farm. This can allow for direct observation of insects, diseases, weeds, and other sources of crop damage. However, farms can be very large in size and physically scouting the entire farm can be time consuming. Remote sensing methods have also been used, such as satellite imaging, spectroscopy, infrared sensors. However, these methods are often affected by post-processing requirements for imaging and spectroscopy and susceptibility of infrared sensors to environmental conditions. Therefore, these methods have been difficult to use in real-time sensing and have limited accuracy.

Many plants release certain volatile organic compounds when the plants are damaged, such as when insects consume a portion of the plants. The systems described herein can be used to detect a volatile organic compound released by damaged plants and to estimate the location of the damage. In some examples, the system can include an array of chemical sensors distributed throughout a field of crops. The sensors can continuously monitor the crops and the sensors can activate if and when a threshold concentration of the target volatile organic compound is detected. The sensors can then send an electronic signal to the source locating module, and the source locating module can estimate the location of the damaged crops that released the volatile organic compounds. Thus, the systems described herein can provide continuous monitoring of crops and near real-time detection of crop damage without a human user physically present in the crop fields Additionally, because the chemical sensors draw little or no power when the concentration of the target volatile compound is below the threshold, the sensors can continuously monitor the crops while consuming a very small amount of electric power.

In further detail regarding the target volatile compounds that can be detected using the present technology, the chemical sensors can be designed to selectively detect a variety of different volatile compounds. It is noted that the chemical sensors can be configured to operate in the air or other gas environments. The volatile compound to be detected can be a gas, vapor, aerosol, particulate, or other matter that can move through the gas environment. In certain examples, the chemical sensor can be configured to respond to individual molecules of the target volatile compound by binding the individual molecules to a binding agent within a nanogap that is specifically sizes to admit an individual molecule of the target volatile compound.

In some examples, the target volatile compound can be a volatile organic compound released by a plant. The volatile organic compound can be released when a plant is damaged, such as by pests, disease, cutting, trampling, underwatering, overwatering, and so on. Non-limiting examples of specific compounds that may be released by damaged plants include hexanal, hexenal, and hexenol. Multiple forms of hexenal and hexenol can be detected, including cis-3-hexen-1-ol, trans-3-hexen-1-ol, 5-hexen-1-ol, 2-hexen-1-ol, 1-hexen-1-ol, trans-3-hexenal, cis-3-hexenal, trans-2-hexenal, cis-2-hexenal, and other forms of hexenal and hexenol. Other non-limiting examples of volatile organic compounds can include 3-diamine, 4-diamine and 5-diamine. In a particular example, hexanal can be a target volatile compound characteristic of mechanical damage to crops, which may be caused by insects or other pests. The chemical sensors can be designed to selectively detect bexanal and send a signal to the source locating module so that the source locating module can estimate the location where the crop damage is occurring In certain examples, the chemical sensor designed to detect hexanal can have electrically conductive structures separated by structure gaps (i.e., nanogaps) with a structure gap distance from 5 nanometers to 6 nanometers. In a particular example, the structure gap distance can be about 5.2 nanometers. A variety of types of crops can release these or other volatile compounds when damaged, or even different compounds based on the type or progress of the damage. Further, in some cases a combination of volatile compounds can be release by damaged crops. The proportion of compounds can also be used to identify the nature and extent of damage. In such cases, the sensors can be configured to include multiple different binding agents on the same, or adjacent sensors, which are targeted to different volatile organic compounds. These multiple sensor array would allow one to obtain a pattern of responses that can be utilized to reduce errors in detection and enable the distinguish of a target chemical with complex structures or a mixture of multiple sources in difference concentrations.

In certain examples, the system can be used to monitor crops such as sorghum, wheat, corn, soybean, potatoes, rice, nuts, cotton, vegetables, and fruits, or a combination thereof. Corresponding volatile organic compounds indicative of plant damage or invasive plant species can thus vary correspondingly. For example, ethylene can indicate levels of bacteria which assist in plant growth and as an indicator of ripeness for apples, apricots, avocados, bananas, kiwi, tomato, and the like. Indole and terpene are indicators of insect damage in sorghum and rice. Acetone, pentanal, 4-methylpentan-2-one, toluene, and dibutyl phthalate are indicators of floral volatiles from parasitic plants such as Orobanche elatior, Orobanche kochii, Orobanche alsatica, Orobanche mayeri, Phelipanche arenaria, Phelipanche ramose, and the like. These volatile organic compound and corresponding plant damage are merely exemplary and other corresponding compounds can be readily detected by corresponding adjustment of the target binding groups.

In further examples, the target volatile compound can be a disease biomarker released by an animal. Disease biomarkers can be characteristic compounds that are given off by animals when the animal has a particular disease. In some types of farms, such as poultry farms and cattle farms, it can be useful to detect infectious diseases as quickly as possible so that infected animals can be removed or isolated to prevent spreading of the disease. The systems described herein can be used to monitor animals for volatile biomarkers of diseases and to identify the animals from which the biomarkers were released. Non-limiting examples of disease biomarkers can include acetaldehyde, decanal, and the like. Table 1 lists a few representative diseases and corresponding volatile compounds indicative of such diseases.

TABLE 1

| Disease | Target VOC |
|---|---|
| Breast Cancer | Methanal (Formaldehyde), 2-amino- |
| | 5-isopropyl-8-methyl-1-azulenecarbonitrile, |
| | 3,3-dimethyl pentane, 5- |
| | (2-methylpropyl)nonane, 2,3,4-trimethyl |
| | decane, 2-Trifluoromethylbenzoic acid, |
| | 6-ethyl-3-octyl ester |
| Lung Cancer | 2-Butanone, butanal, 2-pentanone, |
| | pentanal, hexanal, heptanal, octanal, |
| | acetone, Isobutane, 2,3,4-trimethyl |
| | hexane, 1-hexene, benzene, etbylbenzene, |
| | 1-methyl-4-(1-methylethyl)benzene, |
| | p-xylene, m-xylene, o-xylene, |
| | methanol, isopropanol, 1-propanol |
| SARS-CoV-3 | butyraldehyde |
| Bladder Cancer | Nonanal, isononane, isoprene, |
| | styrene, toluene, ethanol, |
| | 2-ethylhexanol |
| Ovarian Cancer | Decanal, Hexadecane |
| Chronic obstructive | Undecanal, dodecanal, pentadecanal, |
| pulmonary disease | cyclohexanone, 4-methylanisol, hexyl |
| | ethylphosphonofluoridate, indole, 2- |
| | pentylfuran, 6-ethyl-2 methyl |
| | Decane, Oxirane-dodecyl, |
| | 2,4,4-trimethyl-1-pentene, |
| | 1,3,5-tri-tert-butylbenzene, |
| | menthyl acetate, Butylated hydroxytoluene, |
| | Cyclohexanol, phenol, |
| | 2-propanol, Pentanoic acid, |
| | Butanoic acid, Benzofuran |
| Helicobacter | Hydrogen nitrate, ethyl acetate |
| pylori infections | |
| Pseudomonas | Methylthiocyanate, Hydrogen cyanide, |
| aeruginosa | 2-Aminoacetophenone |
| Tuberculosis | Propane, 2-methoxy-2-me , |
| | Cyclohexane, 1,3-dimethyl-, |
| | trans-Cyclohexane, Pentane, |
| | 1,4-dimethyl-Cyclohexane, |
| | 2,4-dimethyl-Heptane, 1-ethyl-4-methyl-, |
| | trans-Cyclohexane, |
| | 3-ethyl-2-methyl-Heptane, 2,6- |
| | dimethyl-Octane, 3-methyl-Heptane, |
| | 4-methyl-Heptane, 4-methyl-Decane, |
| | Tridecane, 1-_beta_-Pinene, |
| | Camphene, 3,6,6-trimethyl- |
| | Bicyclo_3_1_1_hept-2-ene, |
| | 1-Octene, methyl benzene, |
| | 1,4-dichloro benzene, 1,2,3,4- |
| | tetramethyl-Benzene, |
| | ethyl benzene, 1-methyl- |
| | naphthalene, 2-methyl-Styrene, |
| | propyl benzene, 2-butyl-1-octanol |
| Gastric Cancer | Furfural, 6-methyl-5-hepten-2-one, |
| | 2-butoxy-ethanol, 2- |
| | propenenitrile, 2-Ethyl-1-hexanol |
| head and neck | 5-Methyl-3-hexanone, |
| squamous cell | 2,2-Dimethyl-propanoic acid |
| carcinoma | |
| Colon Cancer | 4-(4-propylcyclohexyl)-4′- |
| | cyano[1,1′-biphenyl]-4-yl |
| | ester benzoic acid, 1,3-dimethyl |
| | benzene, 1,1′-(1-butenylidene)bis |
| | benzene, [(1,1-dimethylethyl)thio] |
| | acetic acid, 1-iodo nonane |
| Intra-oral halitosis | Hydrogen sulfide, Methyl mercaptan |
| | (Methanethiol), Dimethyl sulfide, |
| | Dimethyl disulfide, Dimethyl trisulfide |
| Renal failure/uremia | Ammonia |
| Asthma | Nitric oxide, ethane |
| end-stage renal disease | methylene chloride |
| head and neck | Bicyclo[2.2.1]heptane, |
| squamous cell | 2,2,3-trimethyl-, exo-, 4,6- |
| carcinoma | Dimethyl-dodecane, Limonene |
| Crohn's disease | 3-methylhexane |
| irritable bowel disease | 5-ethyl-3-methyloctane |
| end-stage renal disease | nonane |
| Prostate Cancer | 2,2-dimethyl decane |
| Renal failure/uremia | Ethylene |
| Liver Cancer | 2,3-dihydro-benzofuran, acetic |
| | acid, methane-sulfonyl chloride |
| Prostate Cancer | p-xylene |
| Malaria | 3-carene, terpenes, a-pinene |
| diabetes | Methyl Nitrate |
| acute kidney | 2-pentanone |
| injury (AKI) | |
| SARS-CoV-4 | ethyl butanoate |

The systems described herein can also be used to monitor for chemical weapons. Chemical weapons can include nerve agents, blister agents, choking agents, and other types of chemical weapons. In some examples, an array of chemical sensors can be designed to detect one or more of these agents, including volatile organic compounds which are present with these agents. If the target chemical weapon agent is detected then the source locating module can estimate the location of the source, which can help authorities locate the release point of the chemical weapon agent. This can be useful in order to evacuate the area, remove the source of the chemical weapon, and so on. Non-limiting examples of volatile organic compounds associated with chemical- and bio-weapons can include nerve gases of sarin and VX, cyclosarin, mustard gas, and viruses, and the like.

Other target volatile compounds can fit in differently sized structure gaps. In various examples, the chemical sensors can have a structure gap from 0.3 nanometer to 100 micrometers, or from 0.3 nanometer to 1 micrometer, or from 0.3 nanometer to 500 nanometers, or from 1 nanometer to 300 nanometers, or from 1 nanometer to 200 nanometers, or from 1 nanometer to 100 nanometers, or from 1 nanometer to 50 nanometers, or from 1 nanometer to 20 nanometers.

The binding agent can be a compound that can be immobilized on surfaces of the electrodes and electrically conductive structures. The binding agent can also be capable of reversibly binding with the target volatile compound. In some examples, the binding agent can selectively bind the target volatile compound through hydrogen bonding. In certain examples, the binding agent can bind to the target volatile compound through ultra-selective host-guest recognition. This host-guest recognition is the process of holding molecules without covalent (permanent) bonding. A target molecule is adsorbed by a host molecule, such as a crown ether, when the size, shape and charge-distribution of the target and the host match with each other, leading to ultra-specific binding. Since this does not form covalent bonding, the binding can be breakable, and the binding agent thermo-dynamically desorbs the target molecules to reach a lower Gibbs energy equilibrium, enabling reversibility of adsorption. The reversibility depends on adsorption process (instead of absorption) where the target molecules temporarily attach onto the binding sites. As the adsorption period becomes longer, then the reversibility time becomes longer. The length, charges, etc. of the chemical tether can be selected to tune the half-adsorption-lifetime of the receptor complex. In certain examples, the electrodes and electrically conductive structures of the sensor can be treated with amine-PEG-amine and crown-tetracarboxylic-acid layers, forming binding sites for target molecules. In further examples, the binding agent can link to the surfaces of electrodes and electrically conductive structures through a linking group such as a thiol group, an amine group, a siloxy group, or another linking group.

The selection of binding agents can also affect the threshold concentration of the chemical sensors. Binding agents can be selected to have a high degree of conjugation to allow for more electrical conductivity. The length of the binding agent molecules can also be selected to make an appropriately sized space for a single target molecule to be captured between two binding agent molecules. The type of capture group on the binding agent molecule can be selected to match with the target molecule. For example, the capture group can include hydrogen bond donors spaced apart at a distance that matches with hydrogen bond acceptors on the target molecule.

In some examples, the binding agent can have π-conjugated structure and can be electrically conductive. The binding agent can include a core group with a x-conjugated structure and a side binding group that can be modified without altering the electrical properties of the core group. In certain examples, the binding agent can include a sulfhydryl (—SH) group, which can be used to link the binding agent molecule to electrodes and electrically conductive structures of the sensors. The binding agent may also include a second sulfhydryl group to bind to the target volatile compound.

In some examples, the binding agent molecule can have a molecule length from about 1 nm to about 5 nm, or from about 2 nm to about 3 nm. In this chemical sensor, thiol-functional group linker molecules were used to bind the aldehyde functional group chemicals, here hexanal. By using this concept, aldehyde- or hydroxyl-functional-group to bind carboxyl-functional-group chemicals, hydrophobic interaction to bind the alkane-functional-group chemicals, and hydrogen-bonding to bind ester-functional-group chemicals can be utilized for other chemical sensors.

The chemical sensors can operate in a low-power “sleep mode” when the target volatile compound is not present. As explained above, little or no electric current flows across the switch gap of the chemical sensor when the target volatile compound is not at the threshold concentration. Thus, no power is consumed by the positive and negative electrodes when the target volatile compound is not present. In some examples, the chemical sensor can also include a battery and a microcontroller. The microcontroller can normally be shut off or in a low power sleep mode when the target volatile compound is not present. However, the microcontroller can include a “wake-up” circuit that turns on the microcontroller when the target volatile compound reaches the threshold concentration. For example, the wake-up circuit can be connected to the switch gap. When molecules of the target volatile compound bridge the switch gap, electric current can flow across the switch gap. However, the magnitude of the electric current can be fairly small compared to the electric current normally used to run the microcontroller. The wake-up circuit can be triggered by the small current from the switch gap and turn on or wake up the microcontroller for full operation. After this, the microcontroller can perform various functions such as recording the electric current across the switch gap, estimating the concentration of the target volatile compound, sending electronic signals to the source locating module, and others. In some examples, the microcontroller can go back to sleep mode if the concentration of the target volatile compound falls back below the threshold concentration. It is noted that even when the chemical sensor is in “sleep mode” the sensor can still continuously monitor for the presence of the target volatile compound. The positive and negative electrodes can have a continuous voltage applied during sleep mode. Thus, the wake-up circuit can be triggered any time the switch gap is bridged by molecules of the target volatile compound. The chemical sensor can consume little or no power while in sleep mode until the switch gap is bridged. In some examples, an individual chemical sensor in sleep mode can draw standby power from 1 mW to 100 mW, or from 5 mW to 50 mW, or from 10 mW to 25 mW. In some examples, the chemical sensors can be capable of field deployment for a long time, such as greater than 50 days, greater than 100 days, greater than 200 days, greater than 250 days, greater than 300 days, greater than 1 year, or greater than 2 years.

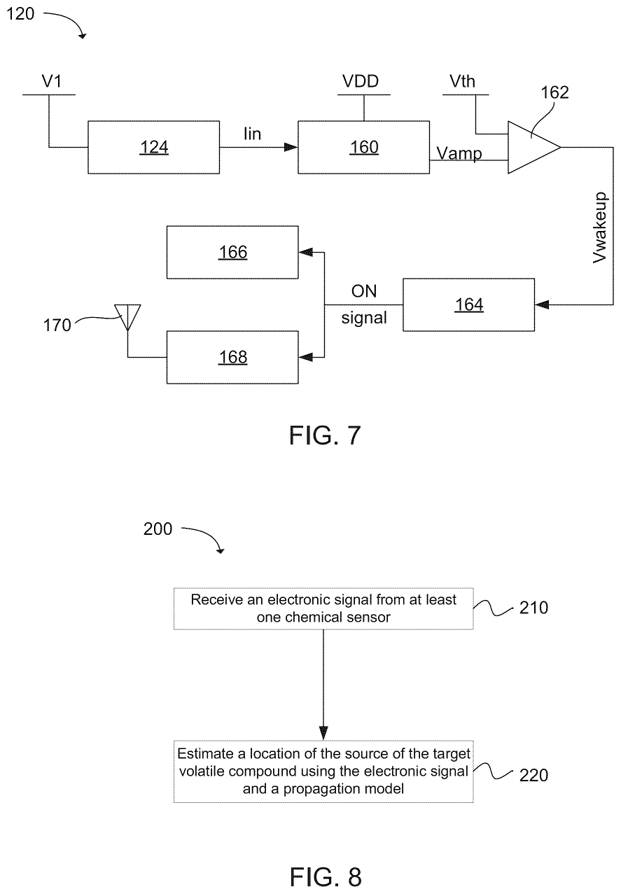

FIG. 7 is a circuit diagram 120 of including another example gas sensor 124. In this figure, the gas sensor 124 is depicted as a box that represents the positive electrode, negative electrode, switch gap, and binding sites in the switch gap. The other boxes in this figure represent other electronic components that are included in this example sensor. These components are electrically connected as shown in the figure. A voltage V1 is applied to the gas sensor. When the gas sensor is exposed to the threshold concentration of the target volatile compound, an electric current Iin crosses the switch gap. This current is fed into a femtoamp amplifier 160. A voltage at drain VDD is also supplied to the femtoamp amplifier. The femtoamp amplifier outputs a higher voltage signal Vamp and this voltage signal is sent to a comparator 162. The comparator compares the Vamp signal to a threshold voltage Vth, and if the Vamp signal is greater than Vth then the comparator sends a wake-up voltage Vwakeup to a microcontroller 164. The microcontroller can be programmed to perform various functions when activated by the wake-up voltage. In this example, the microcontroller turns on an LED display unit 166 and sends a signal through a wireless communication module 168. The wireless communication module sends a wireless electronic signal by way of an antenna 170. This wireless signal can be received by the source locating module.

It is noted that the example shown in FIG. 7 is a single example and other examples may include different electronic components in different arrangements. In some specific examples, a chemical sensor can include one or more of the following components: an amplifier, a comparator, a microcontroller, a battery, a solar panel, an LED, a wireless communication module, an antenna, a wind sensor, a wind-powered generator, and a temperature sensor.

In certain examples, the amplifier can be a femtoamp amplifier configured to amplify an electric current between about I pico-ampere and about 100 pic-amperes. The femtoamp amplifier can be configured to output a voltage signal on the order of millivolts. Example femtoamp amplifiers that can be used include the LMP7721 amplifier available from Texas Instruments Incorporated (United States). In further examples, the comparator can also be a type of amplifier, such as the OPA348 amplifier from Texas Instruments.

In further examples, the chemical sensor can include a microcontroller or other type of electronic controller. The controller can include a processor, memory, and/or other electronic components. Specific examples of microcontrollers that can be used include TEENSY® microcontrollers available from PJRC (Unites States).

The electronic controller can be connected a wireless communication module in some examples. Any type of wireless communication can be used to transmit an electronic signal to the source locating module. In some examples, the wireless communication module can be configured to communicate by radio, Wi-Fi, Bluetooth, cellular network, satellite, or other wireless communication methods. In certain examples, the wireless communication module can be a LORA™ transceiver available from LORA ALLIANCE® (United States).

Any number of chemical sensors can be in communication with the source locating module. The chemical sensors can be arranged in an array in a detection volume. The detection volume can be any volume of interest where a source of the target volatile compound is to be found. For example, if the chemical sensors are used to detect crop damage in a field of crops, then the detection volume can be the air around the crops in the field. When used in a field of crops, the chemical sensors can be arranged in a two-dimensional array spread over the surface of the field. The sensors can be placed at ground level, or at the level of the crops, or above the level of the crops. In other applications, a three-dimensional array of chemical sensors may be useful. For example, a hydroponic farm that has a significant vertical height can be monitored by a three-dimensional array of chemical sensors. The volume monitored by the chemical sensors can be the detection volume. In certain examples, the number of chemical sensors in the system can be from 2 to 1,000, or from 10 to 1,000 or from 10 to 100, or from 100 to 1,000, or from 20 to 100, or from 20 to 80. The chemical sensors can be separated by any desired array spacing. In some examples, the array spacing can be from about 1 meter to about 1,000 meters between chemical sensors, or from about 1 meter to about 500 meters, or from about 1 meter to about 100 meters, or from about 10 meters to about 100 meters, or from about 10 meters to about 500 meters, or from about 100 meters to about 1,000 meters.

The array of chemical sensors can be designed to balance cost of the system with the accuracy of source locating. In examples where the system is used to prevent damage to crops, the economic loss of damaged crops can be balanced against the cost of chemical sensors. A larger number of chemical sensors, which can be more closely spaced in the array, can result in faster and more accurate location of the source of crop damage. This can lead to cost savings by reducing crop damage. On the other hand, increasing the number of chemical sensors increases the cost of the source locating system. Therefore, the size and shape of the array of chemical sensors can be optimized to provide the desirable combination of reduction of crop damage while minimizing or lowering the cost of the source locating system. This optimization can depend on the specific type of crops being farmed, since high-value crops may call for a sensor array with more tightly spaced sensors, but lower-value crops may call for fewer sensors spaced farther apart.

The present disclosure also described methods of locating a source of a volatile compound. These methods can be performed using the systems described above. Additionally, in some examples the methods can be implemented by a computer or multiple computers. In particular, the chemical sensors can include controllers as described above, and the controllers can include one or more processors. The source locating module can also include one or more processors. These processors can be used to perform the methods described herein.

FIG. 8 is a flowchart illustrating one example of a computer implemented method of locating a source of a volatile compound 200. This method includes: receiving an electronic signal from at least one chemical sensor of an array of chemical sensors distributed in a detection volume, wherein individual chemical sensors comprise a positive electrode, a negative electrode separated from the positive electrode by a switch gap, and a binding agent located at a plurality of binding sites in the switch gap, wherein the binding agent is selective for binding to a target volatile compound, wherein the binding sites are distributed in the switch gap such that the binding sites are capable of binding molecules of the target volatile compound to form an electrically conductive pathway via percolation between the positive electrode and the negative electrode when the chemical sensor is exposed to a threshold concentration of the target volatile compound 210; and estimating a location of a source of the target volatile compound using the electronic signal from the at least one chemical sensor and a propagation model of the target volatile compound, wherein the electronic signal is a parameter input into the propagation model 220

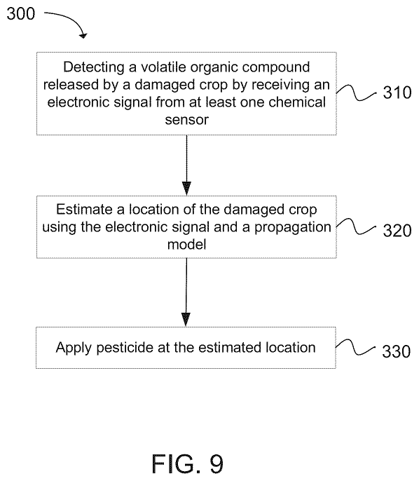

As explained above, one example application for the systems and methods described herein can involve detecting damaged crops that are damaged due to pests. FIG. 9 is a flow chart of an example method of mitigating pest damage to crops 300. This method includes: detecting a volatile organic compound released by a damaged crop by receiving an electronic signal from at least one chemical sensor of an array of chemical sensors distributed in a crop field, wherein individual chemical sensors comprise a positive electrode, a negative electrode separated from the positive electrode by a switch gap, and a binding agent located at a plurality of binding sites in the switch gap, wherein the binding agent is selective for binding to the volatile organic compound, wherein the binding sites are distributed in the switch gap such that the binding sites are capable of binding molecules of the volatile organic compound to form an electrically conductive pathway via percolation between the positive electrode and the negative electrode when the chemical sensor is exposed to a threshold concentration of the volatile organic compound 310; estimating a location of the damaged crop using the electronic signal from the at least one chemical sensor and a propagation model of the volatile organic compound, wherein the electronic signal is a parameter input into the propagation model 320; and applying a pesticide at the estimated location 330.

The source locating module can be programmed to estimate the location of the source of the target volatile compound using a propagation model. The propagation model can vary from simple models to complex. The electronic signal that is received from the chemical sensor can be a parameter input into the propagation model, meaning that the signal is used in some way to help estimate the location of the source. In one example, a simple propagation model can include the assumption that the source of the target volatile compound is located close to the chemical sensor that sends a signal indicating that the threshold concentration of the target compound was detected. Under this model, if a single chemical sensor sends a signal while the other chemical sensors remain quiet, then the location of the source can be estimated at or near the chemical sensor that sent the signal.

In another example propagation model, multiple chemical sensors can be taken into account. For example, if multiple chemical sensors send a signal that the target volatile compound was detected, then the locations of these chemical sensors can be entered as parameters into the propagation model. The propagation model, in this example, may indicate that the source is likely at a location between these multiple sensors Thus, the source locating module can estimate that the source location is between the chemical sensors that detected the target volatile compound.

The propagation model can also include time as a parameter. In one example, if one chemical sensor detects the target volatile compound at a first point in time and then another chemical sensor detects the compound at a later point in time, then it may be assumed that the compound source is nearer to the first chemical sensor than to the second and the rate of diffusion of the compound through the air caused the difference in detection times. Alternatively, it may be assumed that the wind carried the compound to the first chemical sensor and then to the second chemical sensor, and that the source may be upwind of the first chemical sensor.

In further examples, the propagation model can include wind speed and/or wind direction as parameters. The wind speed and wind direction may be measured on-site by a wind sensor that is in electronic communication with the source locating module. The wind sensor can be in the detection volume, or attached to the source locating module, or wind sensor can be incorporated into some or all of the chemical sensors. Alternatively, local weather reporting of the wind speed and direction can be used in the propagation model.

The propagation model can also include other environmental factors, such as temperature. The temperature can be measured by temperatures sensors integrated into the chemical sensors, or a temperature sensor integrated into the source locating module, or a separate temperature sensor in electronic communication with the source locating module. Alternatively, local weather reporting of the temperature can be used.

Similarly, humidity levels can be included in the propagation model. Humidity can also be measured on-site by humidity sensors or local weather reporting of the humidity level can be used.

The propagation model can provide a calculation for the source location of the target volatile compound. Depending on the particular application, the source location can be calculated as a point source, an area source, volume source, or a combination thereof. For example, in a field of crops, the target volatile compound may be released from crops damaged by pests. The pests may be distributed in an area of the field, and thus the source of the target volatile compound can be the entire area where crops are being damaged. In some examples, the propagation model can provide a calculation for estimating the damaged area.

The propagation model can also provide a calculation for estimating transient gas concentrations across a field, and/or the propagation model can take into account measurements of transient gas concentrations across the field. Thus, the propagation model can be used to predict the spread of the target volatile compound across the field. In some examples, the model can include predicting the spread of pests across the field.

The propagation model can also include a calculation of economic loss from damaged crops. The model can be used to estimate economic loss from the damage to crops that has been detected, and/or to predict economic loss from crops that will be damaged as pests spread in the field. The propagation model may also include costs of pesticide and application of treatments to the field. The model can involve a comparison between the economic loss due to damaged crops and the economic cost of treating the field to help determine whether field treatment is desirable.

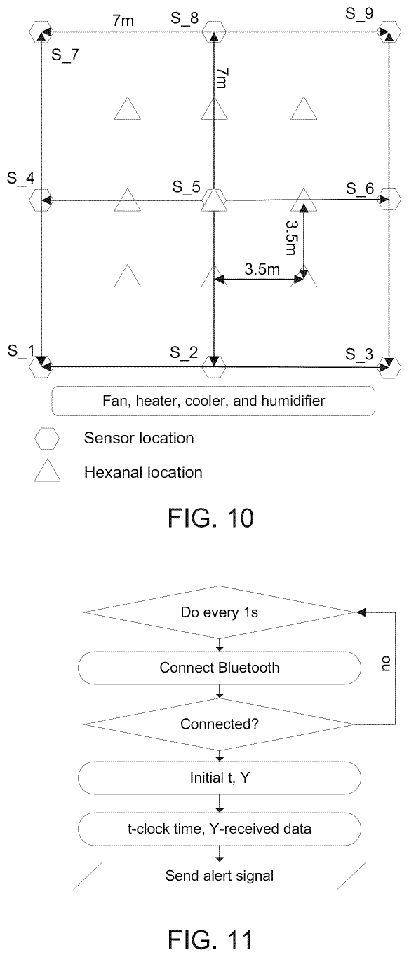

In some examples, propagation models for a particular target volatile compound can be developed by setting up a test sensor array and releasing the target volatile compound from one or more release points within the array, then monitoring the response of the sensors to the target volatile compound. FIG. 10 shows an example test system with release points for hexanal that can be used to develop a propagation model for hexanal. This test system also includes a fan, heater, cooler, and humidifier to provide various simulated weather conditions, which can also be included in the propagation model.

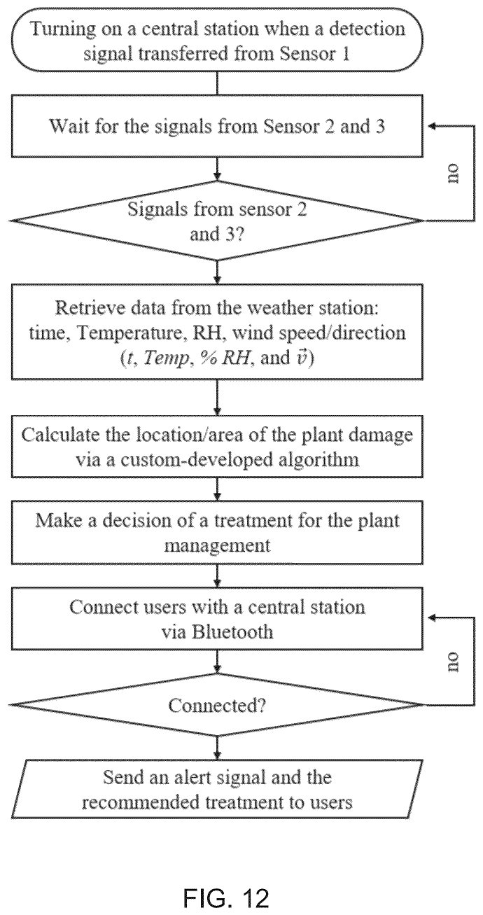

FIG. 11 shows an example algorithm flowchart for wireless communication that can be performed by the chemical sensors when the chemical sensors are turned on. FIG. 12 shows an example algorithm flowchart for an algorithm that can be performed by a volatile compound source locating system. This algorithm begins when a single sensor transmits a detection signal, and includes a series of operations leading up to sending an alert signal and recommended treatment to users. In some examples, the source locating module can perform the calculations involved in this algorithm. These calculations can include any of the features of propagation models described herein.

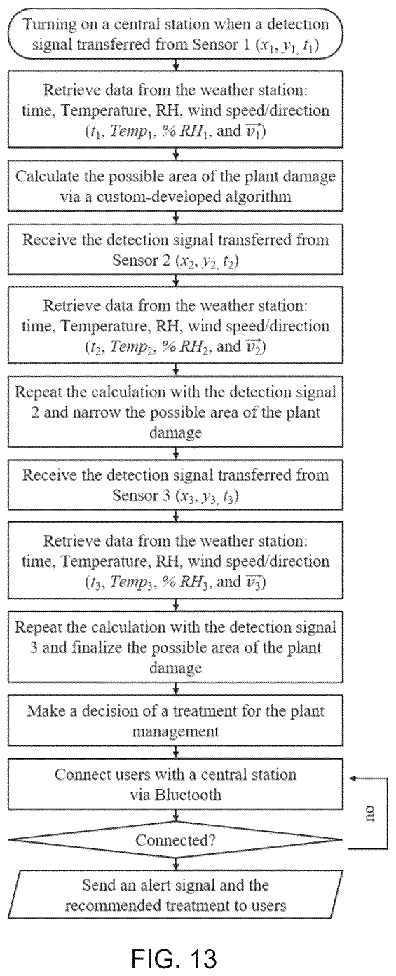

FIG. 13 shows an algorithm flowchart for another example algorithm that can be performed by a volatile compound source locating system as described herein. This algorithm includes calculating possible areas of plant damage multiple times, based on detection signals from multiple different sensors.

### Example 1

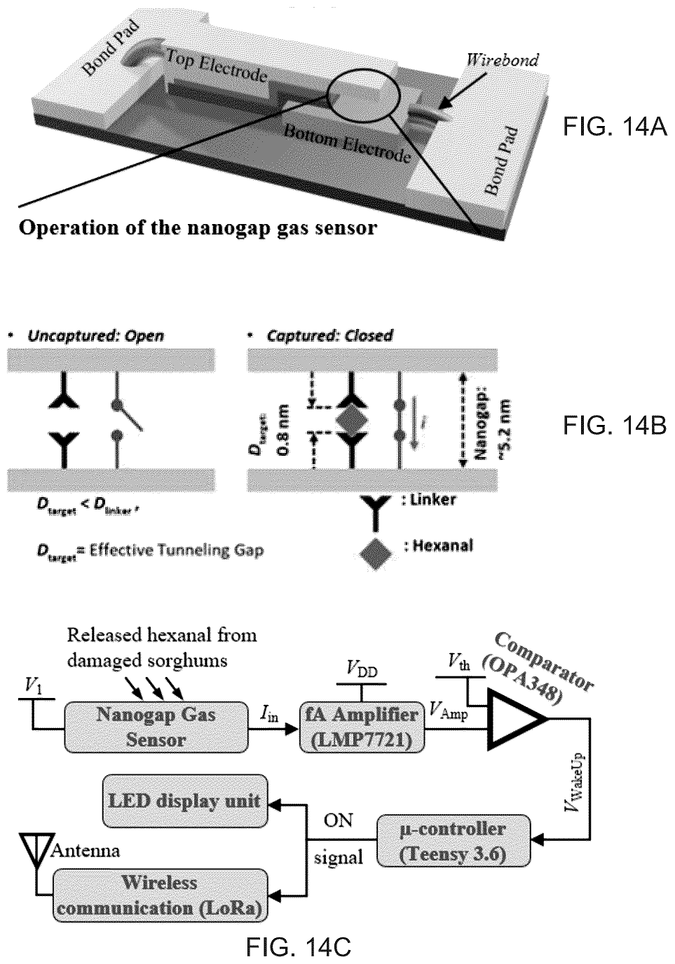

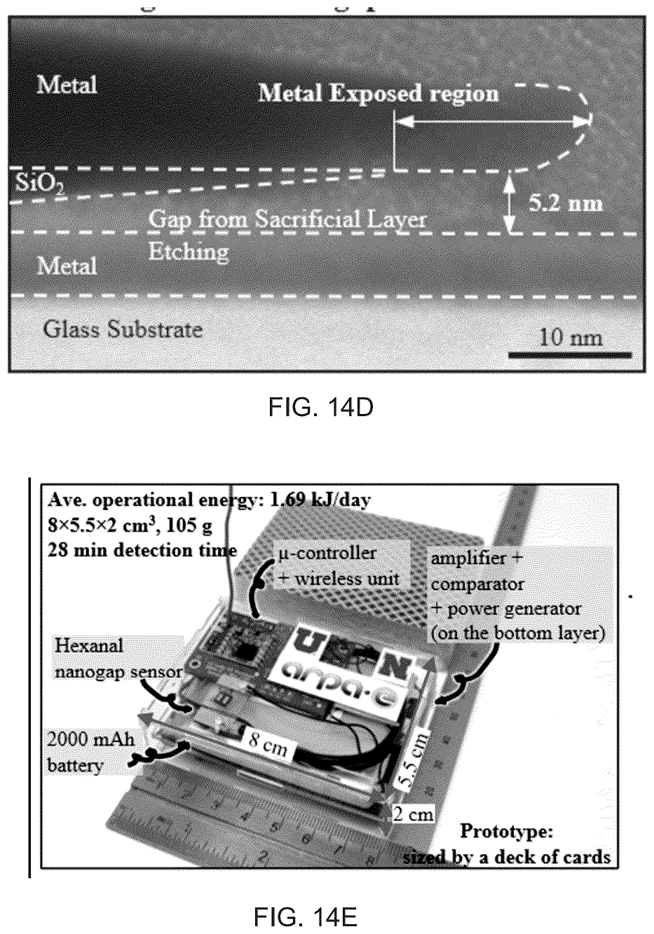

A chemical sensor was constructed to detect hexanal. The sensor had a design as shown in FIGS. 14A-E. The electrodes were formed of gold by sputtering deposition. The nanogap between the electrically conductive structures was about 5.2 nanometers as shown in FIG. 14D. This nanogap size was selected to allow two binding agent molecules with a length of 2-2.5 nanometers and one hexanal molecule with a length of 0.5-0.7 nanometers. The nanogap distance was accurately defined by atomic layer deposition of silicon dioxide and a thin amorphous silicon layer. The nanogap was formed by removing both silicon dioxide and silicon layers via reactive ion etching (RIE). The effective nanogap area was estimated to be about 125 square micrometers and width of about 463 μm.

The prototype chemical sensor included the nanogap gas sensor described above, a fA amplifier, a comparator, a microcontroller, an LED, a wireless communication unit and a battery shown in FIGS. 14C and 14E. The integrated amplifier (LMP7721, Texas Instruments) converted the pico-ampere current from the nano-gap gas sensor into milli-voltage signal. The comparator (OPA348, Texas Instruments) generated 0 V or 4.5 V according to the transmitted voltages from the amplifier. The generated signal was used to determine whether the detection event occurred or not. The microcontroller (Teensy 3.6, PJRC), then, converted the input voltage signals into status of not detected or detected and sent them to the LED and LoRa module. The LED displayed the on-signal at the hexanal capturing only. The LoRa module (RFM95x, Adafruit) transmitted the status signal to the gateway with a frequency of 915 MHz. Finally, the prototype was power-supplied by a LiPo battery (2500 mAh, 3.7V). During the field testing, a successful wireless communication was observed, and the maximum distance between the prototype and the gateway was measured at 72 m.

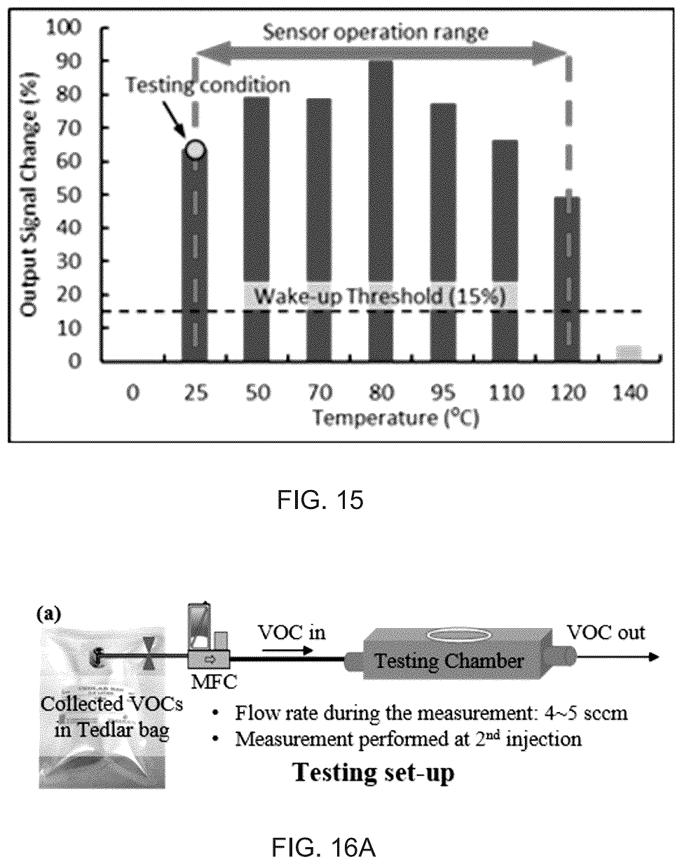

This sensor was tested at different temperatures ranging from 25° C. to 120° C. Within this temperature range, the sensor produced an output signal, which was the ratio resistance, exceeding the level of 1.15 or 15%, which was used as a threshold for electronics wake-up. As the temperature increased from room temperature, the output signal and on/off ratio of resistance rose gradually until 80° C. During this temperature testing, the change in sensor output increased from 63.68% at 25° C. to 90.13% at 80° C., which is a ratio of 1.38 times. Beyond the temperature of 80° C., the sensor output change started to drop, reaching 77.29% at 95° C., 66.41% at 110° C., and finally 49.24% at 120° C. Even at 120° C. with a sensor output change of 49.24%, the sensor is still considered usable since the output change is more than three times the noise value of 15%. These results confirm that increased temperature leads to higher energy and more frequent penetration of the target molecule into the nanogaps until the temperature reaches 80° C. Beyond 80° C. it is likely that damage occurs to the thiol bonding of the chemical linker in the binding agent to the gold surface of the electrodes and electrically conductive structures. This can result in decreasing output signals A graph of the temperature dependence of the output signal is shown in FIG. 15.

### Example 2

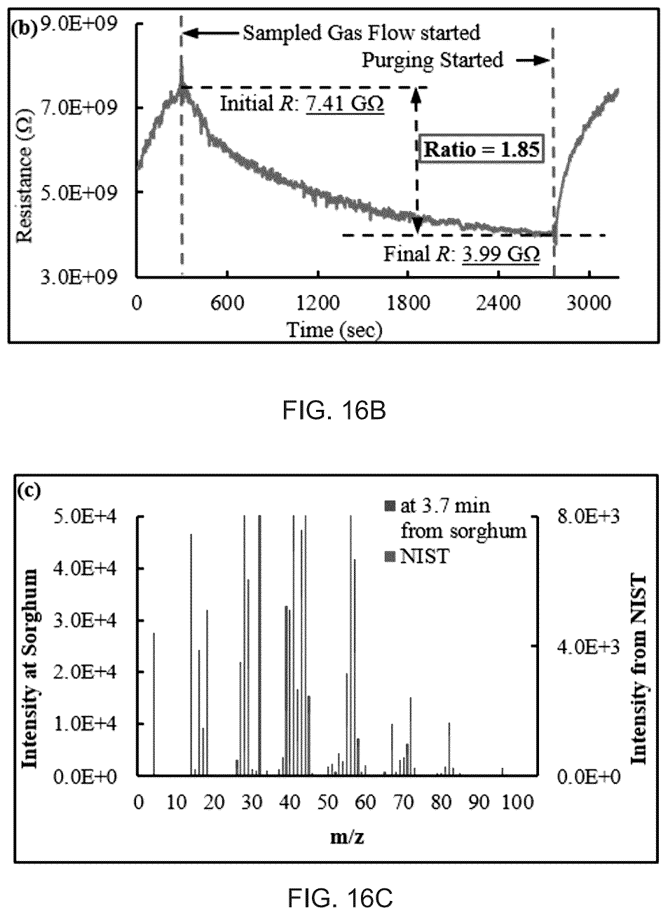

The prototype chemical sensor from Example 1 was tested for in-lab detection of hexanal. Gas samples collected from sorghum leaves were used to test the sensor to determine its response to the hexanal. Sensors were placed in a testing chamber of which the inlet was connected to a tedlar bag containing the prepared gas samples collected from damaged sorghum leaves. A schematic of the test equipment is shown in FIG. 16A. Upon exposure of the gas sample, the sensor output in resistance started decreasing, which indicated the presence of hexanal. A graph of the resistance over time is shown in FIG. 16B. The collected gas samples were then confirmed to contain hexanal by gas chromatography mass spectrometer (GC-MS) analysis. The resultant spectrum from the GC-MS proved a transient peak for hexanal at 3.716 min of the retention time, and the peak was further analyzed via the mass spectrometer (MS) to show mass-to-charge ratios. The mass spectrum (MS) of each peak was compared to the corresponding spectrum in the National Institute of Standards and Technology (NIST) standards. The obtained GC-MS spectrum matched the spectrum of the NIST hexanal standard, confirming that hexanal was released as a dominant volatile compound by damaged sorghum leaves. The GC-MS spectra are shown in FIG. 16C.

### Example 3

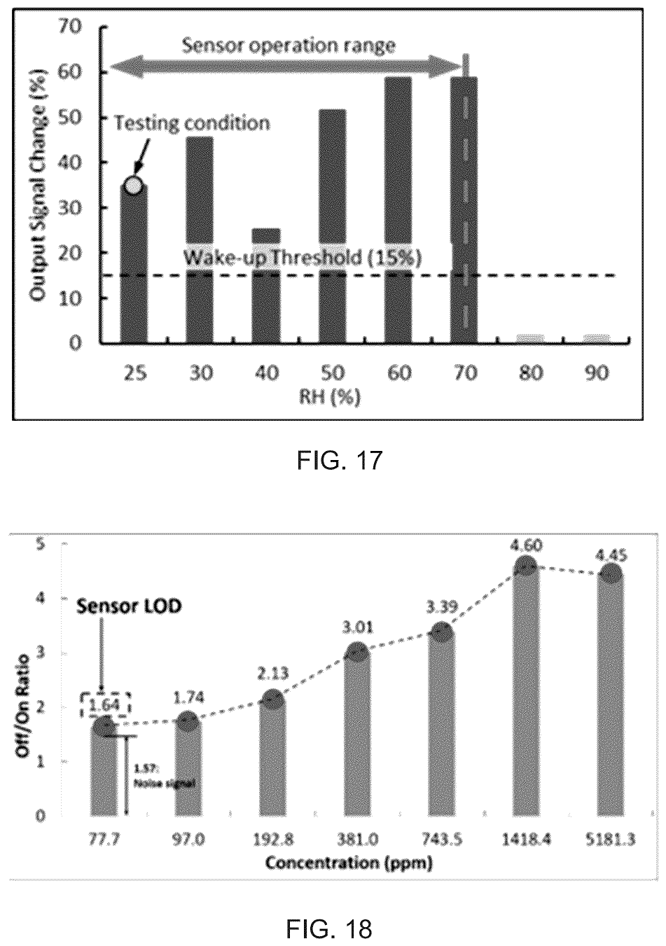

The operational of the sensor was also tested across a range of different humidity levels. The sensor was able to operate at a wide humidity range from 25% to 70%. As the humidity increased, the output remained relatively stable between 30% and 70% humidity except in the relative humidity range of 40%. Above 70% humidity, the sensor response to hexanal was lower than the noise value of 15%. Therefore, the sensor was considered to be unstable at those humidity levels. This may be caused by the hydrogen bond of water molecules near the binding agent molecules, which can inhibit the capture of hexanal. A graph of the sensor output at different humidity levels is shown in FIG. 17.

### Example 4

The sensor was also placed in a micro probe station testing chamber for texting with hexanal. The inlet of the testing chamber was connected to a gas mixing chamber where dilution of pure hexanal vapor occurred. The gas mixing chamber was connected with and fed from two mass flow controllers for the carrier gas (N₂) through a bubbler and for the dilution gas (N₂). The bubbler was composed of a conical flask containing hexanal in a volatile liquid form at room temperature. The hexanal was obtained from Millipore-Sigma. Concentrations of the hexanal vapor in the testing chamber were determined by controlling the volumetric flow rate ratio of carrier gas and the dilution gas with hexanal. Concentrations of hexanal in the bubbler head were estimated as a saturation concentration of 15,384.61 ppm considering a 10 mm Hg vapor pressure of hexanal at atmospheric pressure of 650 mm Hg. The resultant diluted concentrations were a fraction of the saturation concentration depending on the flow rate of the dilution gas. For example, to expose the sensor to a concentration of 77.74 ppm, the carrier gas flow was kept at 5 sccm and the dilution gas flow was at 1,000 sccm. The hexanal concentrations were controlled from 77.73 to 15,384.61 ppm. The sensor output ratio increased with increasing hexanal concentrations up to about 1418.4 ppm. A graph of the sensor output ratio and the concentration of hexanal is shown in FIG. 18.

### Example 5

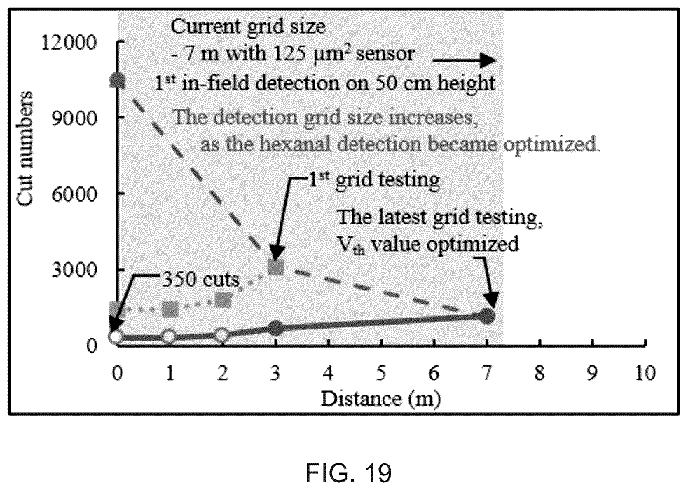

The sensor was tested in a field of 4-month-old sorghum plants. The leaves of sorghum plants near the sensor were continuously cut using scissors at a speed of 50 cuts/minute. When triggered, the gateway station sent an alert message to a database in the central station. The sensor integrated prototype detected hexanal released from mechanically damaged leaves after 7 min˜3.5 hours deployment. FIG. 19 shows a graph of the distance at which hexanal was detected vs. the number of cuts that had been performed. Until the sensor response was obtained, sorghum leaves were continuously cut. Upon detection the prototype displayed a gas detected message on the LED and triggered the LoRa module to send a wireless signal via the gateway, which was observed from 1415 km away. After 20 min˜1.5 hours from the detection, the LED message disappeared indicating sensor recovery in the absence of hexanal During the test only the prototype equipped with a working sensor triggered while other controls that were deployed remained off. This test demonstrated that the sensor was indeed capable of detecting actual hexanal emitted from plants. Field collection of air samples and post-analysis in the standard GC-MS proved the presence of hexanal which was found to be continuously increasing from 29 ppm and to 60 ppm. The accumulation of the hexanal around the prototype during the cutting caused the concentration of hexanal to build up allowing the nano-gap sensor to wake-up the rest of the circuit once a threshold concentration (29 ppm≤C_{threshold}21 60 ppm) was reached.

### Example 6

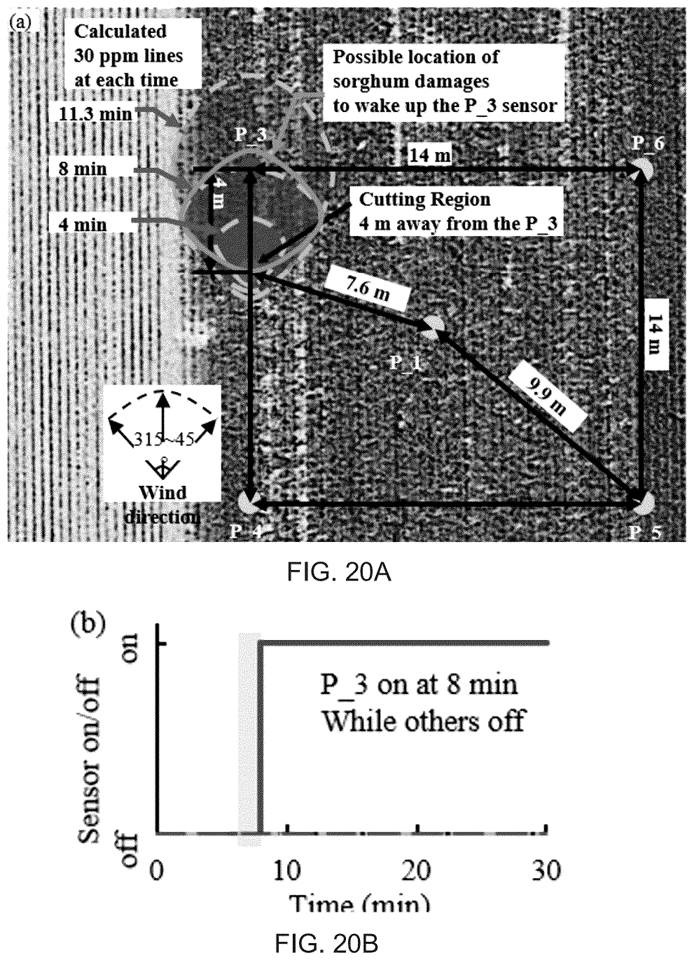

The prototype was tested for pre-localization ability to check the feasibility of array-based detection when sorghums were cut at a known site within an array. Five gas sensor nodes were arranged as shown in FIG. 20A, consisting of four 14-m-away sensors (yellow/blue) in a square configuration and a middle detection point (yellow) within the square. One random spot (red) was selected as the sorghum cut point. The selected point was 4-m away from sensor 3 (P-3, blue) and 10-m away from sensor 4 (P-4, yellow). At the selected point, sorghums were cut at a constant speed of 50 cuts/min. The wind flow in the field was collected from the weather-station database.

As shown in FIG. 20B, after the 8-min period of the sorghum cut, sensor 3 showed an output voltage above a threshold value to send out a detection alert wirelessly. Since sensor 3 was located closest to the cut point, the turn-on of the sensor 3 was as expected. However, other sensors did not respond over a period of 30 min that, in a zero-wind situation, would have turned on sensor 1 in the middle. Later it was found that there was wind blowing toward sensor 3 with an angle of 315˜45° at an average speed of 1.5 mph. Thus, it was hypothesized that the spread of hexanal could not reach sensor 1 for the given period (30 min) of waiting time. During the test, the humidity was 56% RH and the temperature was 38° C.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions and may even be distributed over several different code segments, among different programs and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, a non-transitory machine readable storage medium, such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A volatile compound source locating system, comprising:
an array of chemical sensors distributed in a detection volume, wherein individual chemical sensors comprise a positive electrode, a negative electrode separated from the positive electrode by a switch gap, and a binding agent located at a plurality of binding sites in the switch gap, wherein the binding agent is selective for binding to a target volatile compound based on size, wherein the plurality of binding sites are distributed in the switch gap such that the plurality of binding sites are capable of binding molecules of the target volatile compound to form an electrically conductive pathway between the positive electrode and the negative electrode when the chemical sensor is exposed to a threshold concentration of the target volatile compound; and
a source locating module in electronic communication with the array of chemical sensors, wherein the source locating module is configured to estimate a location of a source of the target volatile compound based on electronic signals from the array of chemical sensors.

2. The system of claim 1, wherein the target volatile compound is a volatile organic compound released by a plant, a disease biomarker released by an animal, or a volatile compound associated with a chemical weapon.

3. The system of claim 2, wherein the target volatile compound is released by a plant and is at least one of hexanal, hexenal, hexenol, acetaldehyde, decanal, diamine, ethylene, indole, terpene, acetone, pentanal, 4-methylpentan-2-one, toluene, and dibutyl phthalate.

4. The system of claim 2, wherein the target volatile compound is released by an animal and is at least one of methanal (Formaldehyde), 2-amino-5-isopropyl-8-methyl-1-azulenecarbonitrile, 3,3-dimethyl pentane, 5-(2-methylpropyl)nonane, 2,3,4-trimethyl decane, 2-Trifluoromethylbenzoic acid, 6-ethyl-3-octyl ester, 2-Butanone, butanal, 2-pentanone, pentanal, hexanal, heptanal, octanal, acetone, Isobutane, 2,3,4-trimethyl hexane, 1-hexene, benzene, ethylbenzene, 1-methyl-4-(1-methylethyl)benzene, p-xylene, m-xylene, o-xylene, methanol, isopropanol, 1-propanol, butyraldehyde, Nonanal, isononane, isoprene, styrene, toluene, ethanol, 2-ethylhexanol, Decanal , Hexadecane, Undecanal, dodecanal, pentadecanal, cyclohexanone, 4-methylanisol, hexyl ethylphosphonofluoridate, indole, 2-pentylfuran, 6-ethyl-2 methyl Decane, Oxirane-dodecyl, 2,4,4-trimethyl-1-pentene, 1,3,5-tri-tert-butylbenzene, menthyl acetate, Butylated hydroxytoluene, Cyclohexanol, phenol, 2-propanol, Pentanoic acid, Butanoic acid, Benzofuran, Hydrogen nitrate, ethyl acetate, Methylthiocyanate, Hydrogen cyanide, 2-Aminoacetophenone, Propane, 2-methoxy-2-me, Cyclohexane, 1,3-dimethyl-, trans-Cyclohexane, Pentane, 1,4-dimethyl-Cyclohexane, 2,4-dimethyl-Heptane, 1-ethyl-4-methyl-, trans-Cyclohexane, 3-ethyl-2-methyl-Heptane, 2,6-dimethyl-Octane, 3-methyl-Heptane, 4-methyl-Heptane, 4-methyl-Decane, Tridecane, 1-_beta_-Pinene, Camphene, 3,6,6-trimethyl-Bicyclo_3_1_1_hept-2-ene, 1-Octene, methyl benzene, 1,4-dichloro benzene, 1,2,3,4-tetramethyl-Benzene, ethyl benzene, 1-methyl-naphthalene, 2-methyl-Styrene, propyl benzene, 2-butyl-1-octanol, Furfural, 6-methyl-5-hepten-2-one, 2-butoxy-ethanol, 2-propenenitrile, 2-Ethyl-1-hexanol, 5-Methyl-3-hexanone, 2,2-Dimethyl-propanoic acid, 4-(4-propylcyclohexyl)-4′-cyano[1,1′-biphenyl]-4-yl ester benzoic acid, 1,3-dimethyl benzene, 1,1′-(1-butenylidene)bis benzene, [(1,1-dimethylethyl)thio] acetic acid, 1-iodo nonane, Hydrogen sulfide, Methyl mercaptan (Methanethiol), Dimethyl sulfide, Dimethyl disulfide, Dimethyl trisulfide, Ammonia, Nitric oxide, ethane, methylene chloride, Bicyclo[2.2.1 ]heptane, 2,2,3-trimethyl-, exo-, 4,6-Dimethyl-dodecane, Limonene, 3-methylhexane, 5-ethyl-3-methyloctane, nonane, 2,2-dimethyl decane, Ethylene, 2,3-dihydro-benzofuran, acetic acid, methane-sulfonyl chloride, p-xylene, 3-carene, terpenes, α-pinene, Methyl Nitrate, 2-pentanone, and ethyl butanoate.

5. The system of claim 1, wherein the plurality of binding sites are distributed to operate the switch gap via percolation wherein a plurality of electrically conductive structures are oriented in the switch gap.

6. The system of claim 5, wherein the plurality of electrically conductive structures are planar islands, vertical pillars or horizontal parallel plates formed in the switch gap.

7. The system of claim 6, wherein the planar islands, vertical pillars or horizontal parallel plates have a shape selected from circular, hexagonal, square, rectangular, and triangular.

8. The system of claim 6, wherein the vertical pillars have a width from 5 nanometers to 100 micrometers and the horizontal parallel plates have a width from 1 micrometer to 1 millimeter.

9. The system of claim 6, wherein the plurality of electrically conductive structures are planar islands having the binding agent attached to surrounding edges of the planar islands.

10. The system of claim 6, wherein the plurality of electrically conductive structures are vertical pillars having the binding agent attached to vertical surfaces of the vertical pillars.

11. The system of claim 6, wherein the plurality of electrically conductive structures are horizontal parallel plates having the binding agent attached to horizontal surfaces of the horizontal parallel plates.

12. The system of claim 11, wherein the target volatile compound is hexanal and wherein the horizontal parallel plates are separated one from another by structure gaps having a structure gap distance from 5 nanometers to 6 nanometers.

13. The system of claim 5, wherein the plurality of electrically conductive structures are separated one from another by structure gaps having a structure gap distance from 0.3 nanometer to 100 micrometers.

14. The system of claim 1, wherein the binding agent includes at least one of an aldehyde-functional group chemicals, thiol-functional-group to bind hexanal-functional-group chemicals, aldehyde- or hydroxyl-functional-group to bind carboxyl-functional-group chemicals, hydrophobic interaction to bind alkane-functional-group chemicals, and hydrogen-bonding to bind ester-functional-group chemicals.

15. The system of claim 1, wherein the chemical sensors are spaced apart one from another at an array spacing from about 1 meter to about 1,000 meters.

16. The system of claim 1, wherein the individual chemical sensors further comprise an amplifier.

17. The system of claim 1, wherein the individual chemical sensors further comprise a microcontroller.

18. The system of claim 1, wherein the individual chemical sensors further comprise a battery.

19. The system of claim 1, wherein the detection volume contains a field of crops and wherein the array of chemical sensors is located such that at least one of the chemical sensors is exposed to the target volatile compound when released from the crops.

20. The system of claim 1, wherein a field of detection is within a field of crops comprising sorghum, wheat, corn, soybean, potatoes, rice, nuts, cotton, vegetables, and fruits, or a combination thereof.

21. The system of claim 1, wherein the source locating module is in electronic communication with the array of chemical sensors through wireless data transmission.

22. The system of claim 1, further comprising a wind sensor in electronic communication with the source locating module.

23. A computer implemented method of locating a source of a volatile compound, comprising:
receiving an electronic signal from at least one chemical sensor of an array of chemical sensors distributed in a detection volume, wherein individual chemical sensors comprise a positive electrode, a negative electrode separated from the positive electrode by a switch gap, and a binding agent located at a plurality of binding sites in the switch gap, wherein the binding agent is selective for binding to a target volatile compound, wherein the plurality of binding sites are distributed in the switch gap such that the plurality of binding sites are capable of binding molecules of the target volatile compound to form an electrically conductive pathway between the positive electrode and the negative electrode when the chemical sensor is exposed to a threshold concentration of the target volatile compound; and
estimating a location of a source of the target volatile compound using the electronic signal from the at least one chemical sensor and a propagation model of the target volatile compound, wherein the electronic signal is a parameter input into the propagation model.

24. The method of claim 23, wherein the plurality of binding sites are distributed to form a percolation-based switch.

25. The method of claim 23, wherein the electronic signal is received from multiple chemical sensors that detected the threshold concentration of the target volatile compound, and wherein locations of the multiple chemical sensors are parameters input into the propagation model.

26. The method of claim 25, wherein the multiple chemical sensors detected the threshold concentration at multiple detection times, and wherein the multiple detection times are a parameter input into the propagation model.

27. The method of claim 23, wherein a wind speed and a wind direction are parameters input into the propagation model.

28. The method of claim 27, further comprising measuring the wind speed and the wind direction within the detection volume.

29. The method of claim 23, wherein the individual chemical sensors draw standby power when not exposed to the threshold concentration of the target volatile compound, wherein the standby power is from 1 mW to 100 mW.

30. A method of mitigating pest damage to crops, comprising:
detecting a volatile organic compound released by a damaged crop by receiving an electronic signal from at least one chemical sensor of an array of chemical sensors distributed in a crop field, wherein individual chemical sensors comprise a positive electrode, a negative electrode separated from the positive electrode by a switch gap, and a binding agent located at a plurality of binding sites in the switch gap, wherein the binding agent is selective for binding to the volatile organic compound, wherein the plurality of binding sites are distributed in the switch gap such that the plurality of binding sites are capable of binding molecules of the volatile organic compound to form an electrically conductive pathway between the positive electrode and the negative electrode when the chemical sensor is exposed to a threshold concentration of the volatile organic compound;
estimating a location of the damaged crop using the electronic signal from the at least one chemical sensor and a propagation model of the volatile organic compound, wherein the electronic signal is a parameter input into the propagation model; and
applying a pesticide at the location.

31. The method of claim 30, wherein the crop comprises sorghum, wheat, corn, soybean, potatoes, rice, nuts, cotton, vegetables, and fruits, or a combination thereof.

32. The method of claim 30, wherein the plurality of binding sites are distributed to form a percolation-based switch.
